(19) United States
(12) Patent Application Publication
(10) Pub. No.: US 2025/0132011 A1
(43) Pub. Date: Apr. 24, 2025

(54) GENERATING ORAL INSERTS ACCORDING TO A TREATMENT PLAN

(71) Applicant: Fresh Health Inc., Mountain View, CA (US)

(72) Inventors: Brent Goldstein, North Las Vegas, NV (US); Kristina Jenna Cook, El Cajon, CA (US); Rebecca Brinkhuis, San Jose, CA (US)

(21) Appl. No.: 18/908,607

(22) Filed: Oct. 7, 2024

Related U.S. Application Data
(60) Provisional application No. 63/591,401, filed on Oct. 18, 2023.

(51) Int. Cl. G16H 20/40; A61C 9/00

(52) U.S. Cl. CPC G16H 20/40; A61C 9/008

(57) ABSTRACT

A treatment plan associated with a user is received. A plurality of oral inserts are designed based on the treatment plan associated with the user, wherein the plurality of oral inserts include a corresponding plurality of fluid nozzles, wherein a corresponding location of one, some, or all of a first plurality of fluid nozzles associated with a first oral insert of the plurality of oral inserts is different than a corresponding location of one, some, or all of a second plurality of fluid nozzles associated with a second oral insert of the plurality of oral inserts. The plurality of oral inserts are generated.

### CROSS REFERENCE TO OTHER APPLICATIONS

This application claims priority to U.S. Provisional Patent Application No. 63/591,401 entitled GENERATING ORAL INSERTS ACCORDING TO A TREATMENT PLAN filed Oct. 18, 2023 which is incorporated herein by reference for all purposes.

### BACKGROUND OF THE INVENTION

Traditional dental and orthodontal work may involve a treatment plan that involves movement of one or more teeth or mouth arches, a change in the number of teeth, or the addition of dental appliances such as braces, retainers, or aligners. It is an enhancement to use a treatment plan to in part predict the oral anatomy of a user. For example, an oral health care device may include an oral insert at least partially customized to the oral anatomy of a user. The user's health care is thus improved if adapted to the treatment plan.

### BRIEF DESCRIPTION OF THE DRAWINGS

Various embodiments of the invention are disclosed in the following detailed description and the accompanying drawings.

FIGS. 1A, 1B, and 1C are schematic depictions of oral anatomy and dental structures, illustrating the regions of the oral cavity described herein.

FIG. 2 is a flow diagram illustrating an embodiment of a process for improving the fit and efficacy of an oral health device for a user in which the user's oral anatomy shifts over a period of time.

FIG. 3 illustrates an embodiment of a perspective view of an oral insert.

FIG. 4 is a diagram of an embodiment illustrating an oral health device.

FIG. 5A depicts a schematic perspective view of a manifold relative to oral anatomy.

FIG. 5B is an illustration of a comparison between a nozzle for a single oral anatomy versus a nozzle for a treatment plan with oral anatomy shift.

FIG. 5C is an illustration of a comparison between a keying feature for a single oral anatomy versus a keying feature for a treatment plan with oral anatomy shift.

FIG. 6 is a functional diagram illustrating a server for improving the fit and efficacy of an oral health device for a user in which the user's oral anatomy shifts over a period of time in accordance with an embodiment.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Generating oral inserts that accommodate a treatment plan for a given user is disclosed. In one embodiment, an oral health device includes a fluid reservoir, a pump, and an oral insert. The oral insert includes a plurality of manifolds and a plurality of fluid nozzles. The oral insert is coupled to the pump. Fluid is introduced into the fluid reservoir. A user may insert the oral insert into their mouth and subsequently activate the oral health device. The plurality of fluid nozzles are at locations customized to the oral anatomy of the user. When the oral health device is turned on, the pump causes fluid to exit the fluid reservoir and to be directed towards the oral anatomy of the user via the plurality of manifolds and the plurality of fluid nozzles. An improvement is that rather than spending two minutes on properly cleaning teeth with a toothbrush and toothpaste, the user spends as little as seven seconds cleaning teeth with the customized oral insert directing fluid to clean their mouth.

Accommodating a dental or orthodontal treatment plan is disclosed. Without accommodation, movement of one or more teeth or mouth arches, a change in the number of teeth, or the addition of dental appliances such as braces, retainers, or aligners may compromise the fit and efficacy of an oral health device with a customized oral insert because the plurality of fluid nozzles may no longer be directed towards the interproximal regions of the user's teeth, reducing the user's health care.

Improving the fit and efficacy of an oral health device for a user in which the user's oral anatomy shifts over a period of time per a treatment plan is disclosed. The user may be wearing or about to start wearing braces or an aligner, and may have difficulties flossing in between their teeth because of the wires associated with braces, which may make the teeth and/or gums less healthy. The location of one, some, or all of the user's teeth and/or the number of user's teeth may change per a treatment plan, which with a static location for an oral insert, may make the teeth and/or gums less healthy. Wires associated with braces may increase the challenge to clean in between the teeth, which if left without resolution may make the teeth and/or gums less healthy. The user may not properly clean brace brackets on a regular basis, such that the user's teeth may have white spots after the braces including brace brackets are removed, which may be unsightly and need better cleaning. Thus, improving the fit and efficacy of the oral health device for a user in which the user's oral anatomy shifts over a period of time per a treatment plan may improve cleaning of the teeth and/or gums, which in turn is an improvement to make the teeth and/or gums cleaner and healthier. That is, using a plurality of oral inserts adapted to the treatment plan may improve cleaning of the teeth and/or gums, which in turn is an improvement to make the teeth and/or gums cleaner and healthier, effectively making the user cleaner and healthier.

In one embodiment, a treatment plan associated with the user is received, for example, an aligner treatment plan. The treatment plan may be electronic in nature, for example, a text-based description/metadata and/or a graphics-based description/metadata, for example, a set of STL files. The treatment plan associated with the user may indicate a series of steps for one, some, or all of the teeth associated with the user. The treatment plan associated with the user may be designed to fix overcrowding of the teeth, a misaligned jaw, an overbite, an underbite, an open bite, and/or a crossbite. The treatment plan may indicate a corresponding amount of movement associated with one, some, or all of the teeth associated with the user, whether the movement may include linear, angular, translational, and/or rotational movement. The treatment plan may also indicate one or more teeth associated with the user that are to be removed and/or predicted to be emerging, for example, an impacted wisdom tooth whose crowding is cleared and thus emerges from gums.

A plurality of oral inserts are designed for the user based on the treatment plan. In one embodiment, an oral insert is designed for a subset of the steps of the treatment plan. For example, each step of the treatment plan may correspond to a visit to an orthodontist's office or the receipt of a new aligner. In another example, an oral insert may be designed to be used for a time period that corresponds to a plurality of orthodontist office visits, for example, six orthodontist office visits at a monthly frequency, or the receipt of a plurality of aligners, for example, receipt of six aligners over a period of six months, each corresponding to a step of the treatment plan.

In one embodiment, an oral insert includes one or more manifolds and/or one or more fluid nozzles. The location of one, some, or all of the plurality of fluid nozzles may change during the treatment plan, that is, the location of one, some, or all of the fluid nozzles associated with a first oral insert may be different than the one, some, or all of the fluid nozzles associated with one or more subsequent oral inserts, to accommodate movement of the teeth.

In one embodiment, an oral insert includes one or more keying features. As referred to herein, a “keying feature” is an oral insert element configured to hold the oral insert in place. The keying feature may be configured to couple the contours of the user's teeth to the oral insert. The location of a keying feature may be added, changed and/or eliminated during the treatment plan.

Fluid Nozzle Directives. In one embodiment, a fluid nozzle is directed downwards towards a gingival margin, an interproximal space between teeth, an incisal edge, or contours of the occlusal, facial, lingual, mesial, and distal surfaces of the teeth. In one embodiment, a fluid nozzle is directed upwards towards a gingival margin, an interproximal space between teeth, an incisal edge, or contours of the occlusal, facial, lingual, mesial, and distal surfaces of the teeth.

In one embodiment, a fluid nozzle is directed towards a gingival margin, an interproximal space between teeth, an incisal edge, or contours of the occlusal, facial, lingual, mesial, and distal surfaces of the teeth at a polar angle between 0° and 180°. In one embodiment, a fluid nozzle is directed towards a gingival margin, an interproximal space between teeth, an incisal edge, or contours of the occlusal, facial, lingual, mesial, and distal surfaces of the teeth at an azimuthal angle between 0° and 180°.

In one embodiment, a fluid nozzle is directed downwards towards a bracket affixed to a tooth of a user, for example, a bracket used in dental braces. In one embodiment, a fluid nozzle is directed upwards towards a bracket affixed to a tooth of a user. In one embodiment, a fluid nozzle is directed towards a bracket affixed to a tooth of a user at a polar angle between 0° and 180°. In one embodiment, a fluid nozzle is directed towards a bracket affixed to a tooth of a user at an azimuthal angle between 0° and 180°.

In one embodiment, the oral insert is designed in a manner such that the flow rate of fluid exiting each of the fluid nozzles is the same within a threshold tolerance. This is to ensure the user has a comfortable experience during a cleaning process, a certain efficacy is maintained during the cleaning process, and/or to ensure safety of the user during the cleaning process. In one embodiment, a number of fluid nozzles associated with a manifold may change during the treatment plan. For example, a user may have one or more teeth extracted during the treatment plan and/or plan to have one or more teeth emerge. One or more flow balancing ports may be added to the one or more manifolds associated with the one or more extracted teeth to prevent uneven fluid flow from the fluid nozzles.

A plurality of different nozzle shapes may be used for a fluid nozzle. For example, a fluid nozzle may be shaped as a circle, a line, a curve, a heart, or arc. A fluid nozzle may have a multi-linear (e.g., V-shaped, T-shaped, X-shaped, etc.) and/or multi-lobular radial shape (e.g., two lobes, three lobes, etc.) and/or irregular shape that is tailored according to the geometry of the user's dental geometry or appliance. A fluid nozzle may have different spray patterns, for example, a full cone pattern, a hollow cone pattern, a flat fan pattern and a solid stream pattern. In one embodiment, the shapes of the fluid nozzles are the same. In one embodiment, the shape of at least one fluid nozzle is different than the shape of the other fluid nozzles. A shape of a fluid nozzle may be selected based on a location of the fluid nozzle within the oral insert. For example, a first shape may be selected to clean the interproximal space between incisors and a second shape may be selected to clear the interproximal space between molars. In one embodiment, a third shape is selected to target brackets, wires, or other dental appliances or restorations.

An expected amount of movement associated with one, some, or all of the teeth may be determined for the plurality of steps of the treatment plan. A degree to which the oral insert is secured when inserted into the user's mouth may also be determined for the plurality of steps of the treatment plan. A shape for a fluid nozzle of the plurality of fluid nozzles may be selected based on the expected amount of movement and/or the degree to which the oral insert is secured. For example, a first shape may be selected in response to a determination that the expected amount of movement associated with a tooth is greater than or equal to a first threshold, i.e., a shape that covers a wide area may be selected. A second shape may be selected in response to a determination that the expected amount of movement associated with a tooth is less than the first threshold, i.e., a shape that covers a narrow area may be selected.

In one embodiment, an oral insert is designed for each step of the treatment plan. For example, an oral insert may be designed to be used for a time period associated with consecutive orthodontist office visits or a time period associated with the receipt of a first aligner and a second aligner. In one embodiment, the plurality of oral inserts are generated according to the design and provided to the user.

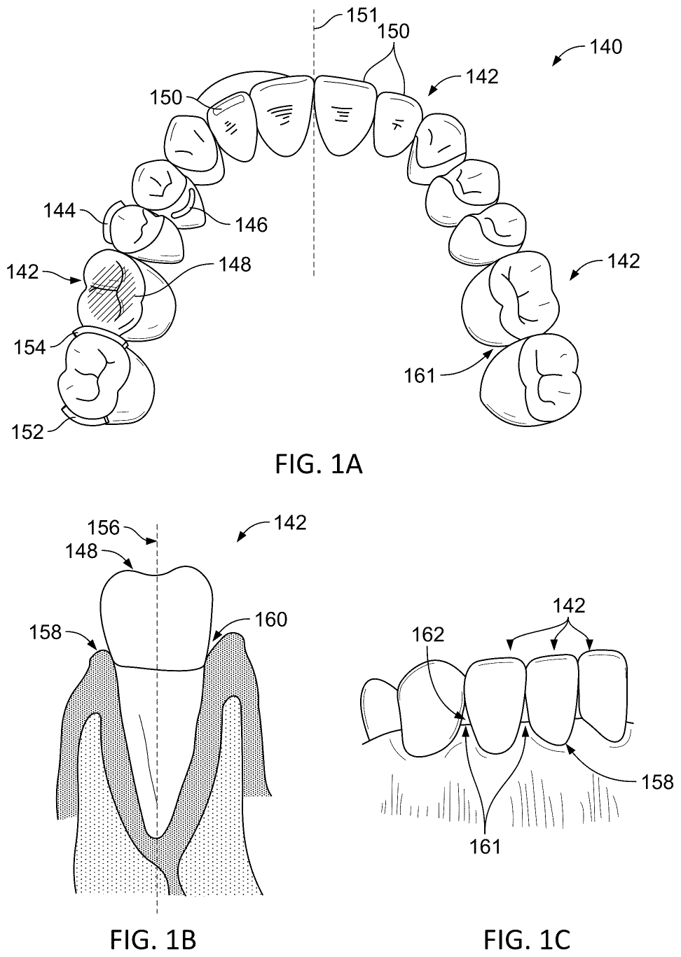

FIGS. 1A, 1B, and 1C are schematic depictions of oral anatomy and dental structures, illustrating the regions of the oral cavity described herein. FIG. 1A depicts a top view of a set of teeth (140) of the mandible or lower jaw, though similar terminology may be used to refer to the teeth and structures of the maxilla or upper jaw. Each tooth (142) may have a facial surface (144) which is the region of the tooth that contacts the cheeks or lips and a lingual surface (146) which is the region of the tooth that contacts, or is nearest to, the tongue. Facial surfaces may be, for example, the buccal surfaces of the posterior teeth and the labial surfaces of the anterior teeth. Lingual surfaces may also be referred to as the palatal surfaces for maxillary teeth. Posterior teeth may have an occlusal surface (148) and the anterior teeth may have an incisal edge or surface (150). The occlusal, or incisal, surface is the region of the tooth that aids in chewing, and/or faces across from the occlusal, or incisal, surface of the opposing tooth. The surface of a tooth facing away from the arch midline may be referred to as the distal surface (152) while the surface of a tooth facing toward the arch midline (151) may be referred to as the mesial surface (154).

FIG. 1B depicts a side view of a single tooth (142), which may have a longitudinal axis (156) that extends along the longest dimension of the tooth (142) and/or is substantially perpendicular to the occlusal surface (148) or incisal edge (150) of the tooth. The edge or boundary of the gums (for example, gingiva/gingival tissue) along the surfaces of the teeth or closest to the occlusal surfaces or incisal edge of the teeth may be referred to as the gingival margin (158). The gingival margin (158) may have one or more curves along the bottom of each tooth, and the radius of curvature and length of the gingival margin for each tooth may vary. A space or region (160) between the gingiva and the surfaces of the tooth may be referred to as a gingival sulcus (160). Interdental gingiva (161) may be the gum tissue located between two adjacent teeth.

FIG. 1C depicts a side view of a plurality of teeth (142). The space or gap between each tooth (142) may be referred to as the interproximal space or gap (162), and may be defined by the mesial surface of one tooth and the distal surface of the adjacent tooth, or the mesial surfaces of two teeth, in the case of central incisor teeth. The left side of a user's oral cavity may be the region of the oral cavity that is to the left of the interproximal space between the two central incisors (for example, to the left of the arch midline), and the right side of a user's oral cavity may be the region of the oral cavity that is to the right of the interproximal space between the two central incisors (for example, to the right of the arch midline).

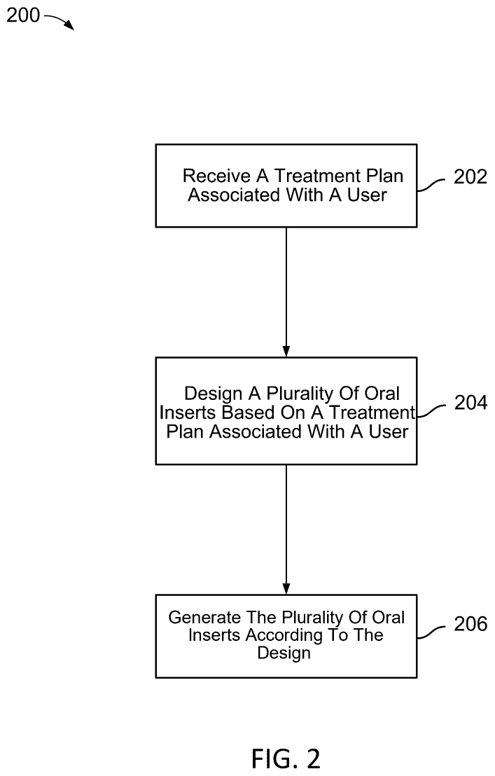

FIG. 2 is a flow diagram illustrating an embodiment of a process for improving the fit and efficacy of an oral health device for a user in which the user's oral anatomy shifts over a period of time. In the example shown, process (200) may be implemented by a server, such as server (600).

At step (202), a treatment plan associated with a user is received. The user may be wearing or about to start wearing braces or an aligner. The treatment plan associated with the user indicates a series of steps for one, some, or all of the teeth associated with the user. The treatment plan associated with the user may fix overcrowding of the teeth, a misaligned jaw, an overbite, an underbite, an open bite, and/or a crossbite. The treatment plan may indicate a corresponding amount of movement, for example, linear, angular, translation, and/or rotational, associated with one, some, or all of the teeth associated with the user.

At step (204), a plurality of oral inserts are designed based on the treatment plan associated with the user. In some embodiments, an oral insert is designed for a subset of the steps of the treatment plan. Each step of the treatment plan may correspond to a visit to an orthodontist's office or the receipt of a new aligner. An oral insert may be designed to be used for a time period that corresponds to a plurality of orthodontist office visits (for example, six orthodontist office visits at a monthly frequency) or the receipt of a plurality of aligners (for example, receipt of six aligners over a period of six months).

An oral insert includes a plurality of manifolds and a plurality of fluid nozzles. The location of one, some, or all of the plurality of fluid nozzles may change during the treatment plan, that is, the location of one, some, or all of the fluid nozzles associated with a first oral insert may be different than the one, some, or all of the fluid nozzles associated with a second oral insert.

In one embodiment, a fluid nozzle is directed downwards towards a gingival margin, an interproximal space between teeth, an incisal edge, or contours of the occlusal, facial, lingual, mesial, and distal surfaces of the teeth. In one embodiment, a fluid nozzle is directed upwards towards a gingival margin, an interproximal space between teeth, an incisal edge, or contours of the occlusal, facial, lingual, mesial, and distal surfaces of the teeth. In one embodiment, a fluid nozzle is directed towards a gingival margin, an interproximal space between teeth, an incisal edge, or contours of the occlusal, facial, lingual, mesial, and distal surfaces of the teeth at a polar angle between 0° and 180°. In one embodiment, a fluid nozzle is directed towards a gingival margin, an interproximal space between teeth, an incisal edge, or contours of the occlusal, facial, lingual, mesial, and distal surfaces of the teeth at an azimuthal angle between 0° and 180°. In one embodiment, a fluid nozzle is directed to target brackets, wires, lingual bars, or other dental appliances or restorations.

In one embodiment, a fluid nozzle is directed downwards towards a bracket affixed to a tooth of a user. In one embodiment, a fluid nozzle is directed upwards towards a bracket affixed to a tooth of a user. In one embodiment, a fluid nozzle is directed towards a bracket affixed to a tooth of a user at a polar angle between 0° and 180°. In one embodiment, a fluid nozzle is directed towards a bracket affixed to a tooth of a user at an azimuthal angle between 0° and 180°.

The oral insert is designed in a manner such that the flow rate of fluid exiting each of the fluid nozzles is the same within a threshold tolerance. This is to ensure the user has a comfortable experience during a cleaning process, a certain efficacy is maintained during the cleaning process, and/or to ensure safety of the user during the cleaning process. In one embodiment, a number of fluid nozzles associated with a manifold may change during the treatment plan. For example, a user may have one or more teeth extracted during the treatment plan. One or more flow balancing ports may be added to the one or more manifolds associated with the one or more extracted teeth to prevent uneven fluid flow within a certain tolerance or range from the fluid nozzles.

A plurality of different nozzle shapes may be used for a fluid nozzle. For example, a fluid nozzle may be shaped as a circle, a line, a curve, a heart, or an arc. A fluid nozzle may have a multi-linear (e.g., V-shaped, T-shaped, X-shaped, etc.) and/or multi-lobular radial shape (e.g., two lobes, three lobes, etc.) and/or irregular shape that is tailored according to the geometry of the user's dental geometry or appliance. A fluid nozzle may have different spray patterns, for example, a full cone pattern, a hollow cone pattern, a flat fan pattern, and a solid stream pattern. In one embodiment, the shapes of the fluid nozzles are the same. In one embodiment, the shape of at least one fluid nozzle is different than the shape of the other fluid nozzles. A shape of a fluid nozzle may be selected based on a location of the fluid nozzle within the oral insert. For example, a first shape may be selected to clean the interproximal space between incisors and a second shape may be selected to clear the interproximal space between molars. In one embodiment, a third shape is selected to target brackets, wires, or other dental appliances or restorations.

An expected amount of movement associated with one, some, or all of the teeth may be determined for the plurality of steps of the treatment plan. A shape for a fluid nozzle of the plurality of fluid nozzles may be selected based on the expected amount of movement. For example, a first shape may be selected in response to a determination that the expected amount of movement associated with a tooth is greater than or equal to a first threshold, that is, a shape that covers a wide area may be selected. A second shape may be selected in response to a determination that the expected amount of movement associated with a tooth is less than the first threshold, that is, a shape that covers a narrow area may be selected.

In one embodiment, an oral insert is designed for each step of the treatment plan. For example, an oral insert may be designed to be used for a time period associated with consecutive orthodontist office visits or a time period associated with the receipt of a first aligner and a second aligner.

At step (206), the plurality of oral inserts are generated according to the design. The server may be coupled to a 3D printer. The design may be provided to a 3D printer. In response, the 3D printer generates the plurality of oral inserts.

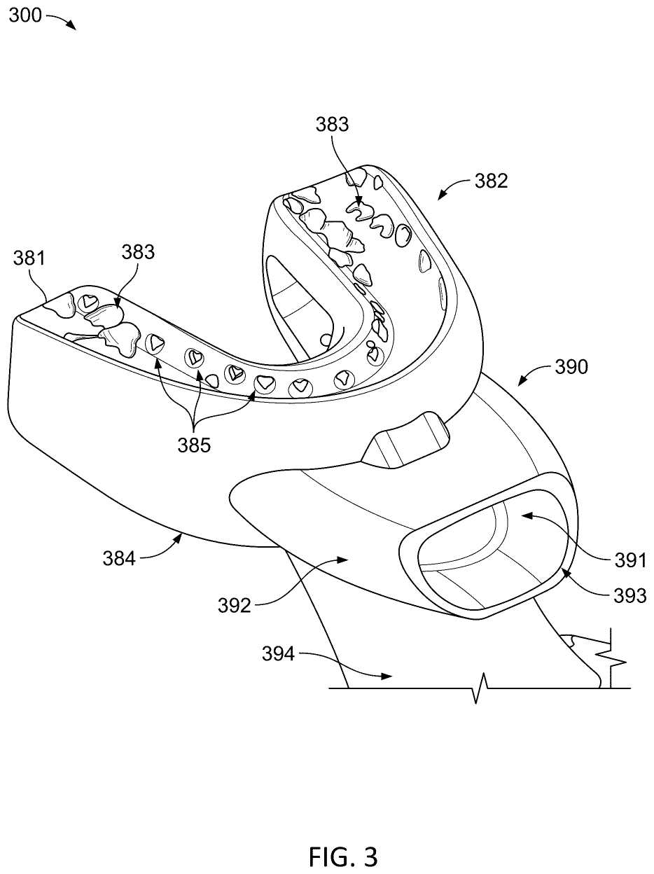

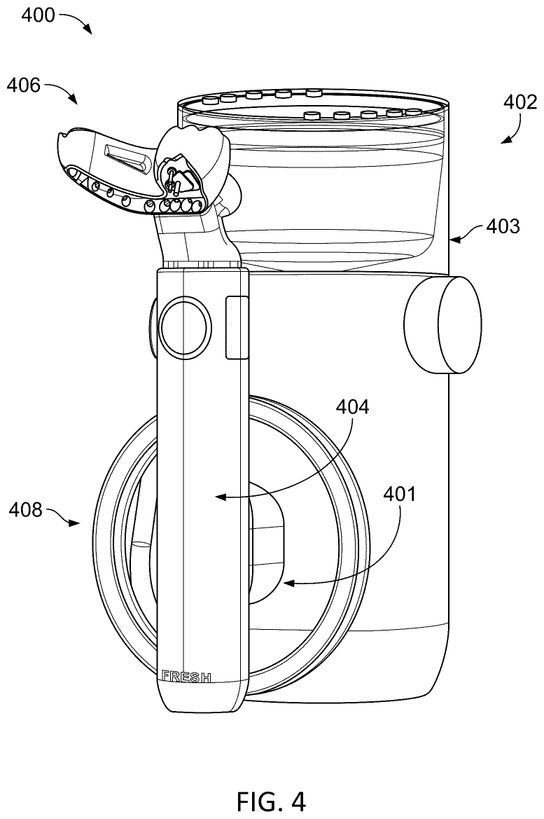

FIG. 3 illustrates an embodiment of a perspective view of an oral insert. The oral insert (300) may be made of a rigid material such as a hard resin compatible with 3D printing, and may comprise an upper portion (382) having a tray configured to receive a user's upper teeth, a lower portion (384) having a tray configured to receive a user's lower teeth, a plurality of fluid nozzles located in the upper portion and the lower portion, and an effluence conduit (390) located between the upper portion and the lower portion. The trays of the upper and lower portions may comprise one or more alignment features (383) including keying features, which may comprise protrusions, slots, or recesses that receive and/or articulate with the user's teeth, gums, hard palate, soft palate, other oral structures, and/or may have contours that correspond to one or more teeth. These alignment features may help to ensure that the oral insert is seated in a desired position in the user's mouth.

In one embodiment, one or more of the fluid nozzles may be located in recesses or indentations (385) along the teeth—and/or gingiva-facing surfaces of the upper and lower trays. The recesses (385) and the fluid nozzles may be located at regions of the upper and lower trays that correspond with the interproximal spaces between the user's teeth, and/or at locations that allow the fluid nozzles to direct fluid jets to the interproximal spaces, for example, that may not necessarily correspond with the locations of the interproximal spaces. The recesses (385) may have a flared or tapered shape, and/or may have one or more concave contours, where the fluid nozzle opening is located at the narrow portion of the recess. The width of the recess may increase as it extends outward from the fluid nozzle opening. The increased width of a flared recess may allow the fluid jet spray to expand unimpeded.

In one embodiment, the central axis of the flared recess may be aligned with the direction of the fluid jet. The effluence conduit (390) may comprise a central port or channel (391) which may extend between a posterior region and anterior region of the oral insert, and may protrude forward at the anterior region as a beak or an elongated spout (392) that terminates at a fluid egress opening (393). The central port or channel (391) is configured to have a minimum cross-section area that causes the fluid outputted from effluence conduit (390) to have a particular pressure. The minimum cross-section area is needed to prevent a high back-pressure that would cause discomfort for the user, for example, a choking/gag feeling, when using oral insert (300).

In one embodiment, alternatively or additionally, an effluence conduit comprises a first side fluid cavity or channel and a second side fluid cavity or channel. The side fluid cavities may funnel into the central port, or may each have their own elongated spouts with separate fluid egress openings. The shape, sizing, and surface contours of the effluence conduit may be configured according to the user's oral anatomy, for example, size and size of oral cavity, location of teeth, and so on, and configured to promote fluid dynamic efficiency in draining the fluid from the user's mouth. In one embodiment, the fluid delivered to the user's mouth may be pressurized and/or delivered at a high fluid rate in order to effectively clean their teeth and/or dislodge particles trapped in the interproximal spaces. Because of the increased rate and/or pressure of fluid flow into the oral cavity/fluid ingress, the effluence conduit of the oral insert may be sized and shaped to allow for fluid egress at the same or greater rate as fluid ingress.

In one embodiment, the oral insert (300) also comprises one or more fluid manifolds, which may be a series of branched and/or networked internal fluid manifolds that distribute the fluid from the handle to the individual fluid nozzles. The fluid manifolds may terminate at a series of manifold openings in a manifold connector port (394) of the oral insert. In one embodiment, the manifold connector has one opening per fluid manifold, or has more than one manifold opening per fluid manifold. When the oral insert is connected to the handle, the fluid regulator components may be configured to fluidically engage with the manifold connector such that the fluid regulator controls the fluid flow into the manifolds. Optionally, the fluid regulator in the handle may further comprise a manifold block having a plurality of fluid channels that are configured to be aligned with the manifold openings in the manifold connector.

FIG. 4 is a diagram of an embodiment illustrating an oral health device. In the example shown, oral health device (400) includes base station (402) that is coupled to fluid reservoir (403), handle (404), and a customized oral insert (406) coupled to handle (404). Oral insert (406) may comprise an upper portion and a lower portion that are sized and shaped according to a user's oral cavity. A plurality of fluid nozzles of oral insert (406) are directed toward interproximal spaces between the user's teeth. Oral insert (406) further comprises an effluence conduit located between the upper portion and the lower portion and configured to channel fluid circulating within the user's mouth to an anterior or facial region of the user's oral cavity. One or more fluid conduits (408) may connect fluid reservoir (403) to handle (404), which has a fluid regulator that distributes fluid to oral insert (406). System (400) may optionally comprise charging station (401) for handle (404).

Handle (404) may comprise one or more control buttons, for example, a start/stop button, and/or a fluid flow adjustment dial/settings selector. In one embodiment, the one or more control buttons may be located on base station (402). In one embodiment, charging station (401) and handle (404) are optional and oral insert (406) is coupled directly to fluid reservoir (403) via the one or more conduits (408). In one embodiment, handle (404) includes a motor associated with a manifold switch.

The oral insert (406) is customized to a user. That is, oral insert (406) includes a plurality of fluid nozzles that are arranged in accordance with the unique geometry of the user's oral cavity, gingival geometry, dental structures, and any oral and/or dental devices or implants. Examples of oral and/or dental devices may include, but are not limited to, permanent and removal dental restorations/prosthetics, orthodontic appliances, crowns, bridges, implants, braces, retainers, and/or dentures. Each of the fluid nozzles is positioned to target a specific dental feature. Inside of oral insert (406), the fluid nozzles may be connected to one or more internal manifolds. The inlets of these manifolds may extend from the back of oral insert (406) in the form of a standardized connector, to which handle (404) and/or conduit (408) may be connected.

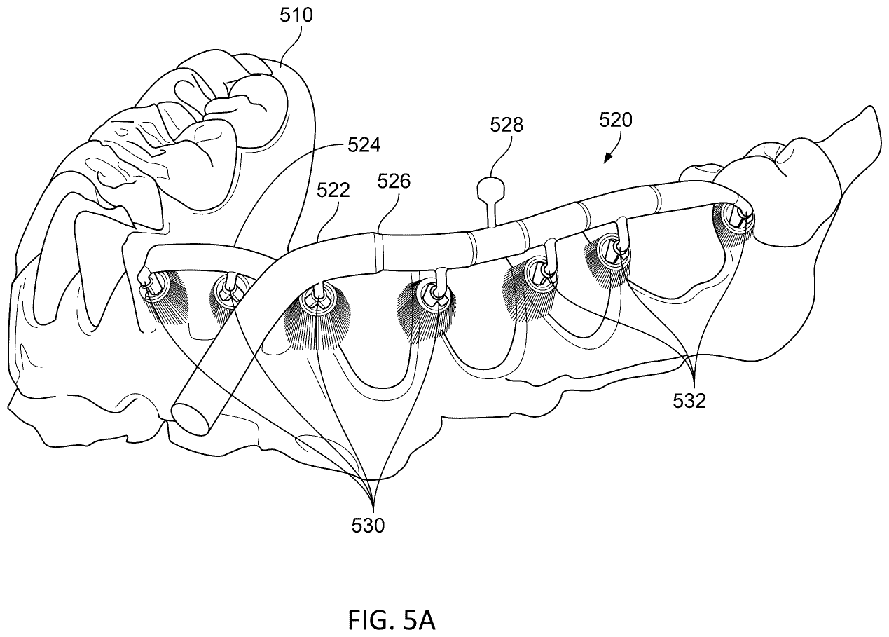

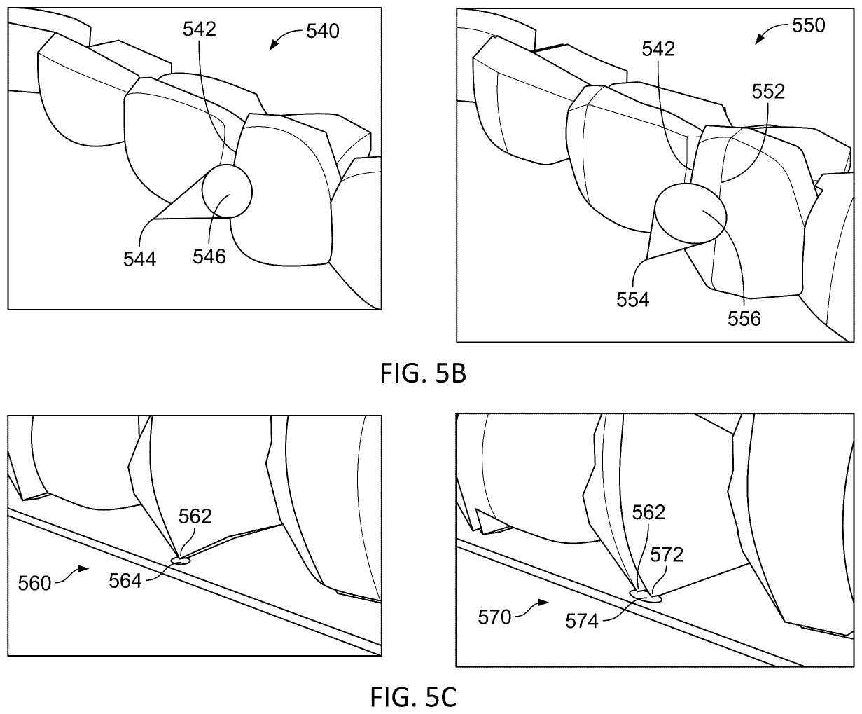

FIG. 5A depicts a schematic perspective view of a manifold relative to oral anatomy (510). The manifold (520) may be coupled to a set of fluid nozzles (530) comprising a set of fluid clearance geometries (532). The manifold (520) may comprise one or more of a trunk (522), branch (524), step region (526), and flow balancing port (528). The trunk (522) may be a primary fluid conduit coupled between a fluid inlet port (not shown) and each of a branch (524), flow balancing port (528), and fluid nozzle (530). The trunk (522) may comprise a larger diameter than any of the branch (524), flow balancing port (528), and fluid nozzles (530). The branch (524) may extend from the trunk (522) and comprises a diameter smaller than the trunk (522).

The branch (524) may serve as a secondary fluid conduit extending from a trunk (522) for fluid nozzles (530) not otherwise coupled to the trunk (522). For example, a branch (524) may extend from the trunk (522) in FIG. 5A for fluid nozzles (530) corresponding to a set of anterior teeth of a patient. Branches may be useful to reduce tortuosity, for example, to prevent kinks in the manifold (520) and improve hydraulic performance of the oral insert. In one embodiment, one or more flow balancing ports (528) may extend from one or more of trunks (522) and branches (524). A flow balancing port (528) may be configured to release pressure as fluid flows through the manifold (520). Without flow balancing ports (528), the manifold (520) and fluid nozzles (530) both may generate uneven fluid flow from the fluid nozzles (530).

In one embodiment, a flow balancing port (528) may be useful when a number of nozzles on all manifolds is not equal in order to maintain a predetermined range of hydraulic pressures. For example, a first manifold may include eight fluid nozzles and a second manifold may include seven fluid nozzles and one flow balancing port. The location and size of the pressure port may be designed such that a flow rate of fluid exiting the eight fluid nozzles associated with the first manifold is the same flow rate of fluid exiting the seven fluid nozzles associated with the second manifold.

In one embodiment, a diameter of the manifold (520) may decrease along a length of the manifold (520) due to pressure relieved by a flow balancing port (528) on the manifold (520). In one embodiment, the diameter of the manifold (520) decreases in a continuous manner. The diameter of the manifold (520) may decrease at a particular angle. In one embodiment, the diameter of the manifold (520) decreases in a stepped manner. For example, the diameter of the manifold (520) may have a first diameter between a first fluid nozzle and a second nozzle and a second diameter between the second nozzle and a third nozzle.

In one embodiment, the number, shape, size, and location of the flow balancing port (528) may be configured to promote a predetermined range of pressures through at least a portion of the manifold (520). For example, one or more flow balancing ports (528) may be configured such that a fluid pressure within the manifold (520) may be within a predetermined range, for example, relatively constant, throughout a length of the manifold (520). In one embodiment, a flow balancing port (528) may be located downstream of a step region (526) of the manifold (520). In one embodiment, the length of at least one manifold is different than the other manifolds. The flow rate of fluid exiting a fluid nozzle on each of the plurality of manifolds is however preferred to be the same within a predetermined threshold tolerance. One or more flow balancing ports may be added to a manifold to increase a flow rate associated with the fluid nozzles associated with the manifold to match the flow rate of the fluid nozzles associated with the other manifold(s).

FIG. 5B is an illustration of a comparison between a nozzle for a single oral anatomy versus a nozzle for a treatment plan with oral anatomy shift. For a single oral anatomy in perspective view (540), there is an interproximal space between a user's two teeth (542) that would be cleaned by a nozzle (544) with a conical spray pattern (546) landing in a roughly circular pattern in the interproximal space (542). For a treatment plan with oral anatomy shift in perspective view (550), there is a shift in the interproximal space between the user's two teeth that shifts from a first space (542) to a second space (552). For the treatment plan flexible fluid nozzle (554) to cover both the first space (542) and the second space (552), a more broad nozzle spray such as a wider conical spray pattern (556) and/or fan pattern (not shown) is used landing in a roughly elliptical pattern in both interproximal spaces (542), (552).

Thus, with a less precise nozzle shape and/or more broad nozzle spray pattern (556), more flexibility with oral anatomy shift may be tolerated and/or the oral insert is more forgiving. In one embodiment, with a treatment plan oral insert, the system encourages the user to set the fluid flow settings to increase rinse duration, rinse intensity, and/or rinse pulsation rate to compensate for the less precise spray pattern (556). In one embodiment, with a treatment plan oral insert, the system encourages the user to use a treatment rinse solution to aid in cleaning a bracket, wire, or attachment associated with an orthodontic appliance such as braces, for example, one with more intense ingredients. In one embodiment, with a treatment plan oral insert, the system encourages the user to use an after treatment rinse solution to aid in whitening and/or reducing discoloration/white spots after braces including when brace brackets are removed/buildup occurs around a bracket.

Not shown in FIG. 5B, if as part of a treatment plan a tooth is removed and/or an interproximal space is removed, the nozzle may itself be removed, referred to herein as a no [symbol] le absence. Not shown in FIG. 5B, if as part of a treatment plan a tooth is planned to emerge, a nozzle may be added to the oral insert, referred to herein as a no [symbol] le addition.

FIG. 5C is an illustration of a comparison between a keying feature for a single oral anatomy versus a keying feature for a treatment plan with oral anatomy shift. For a single oral anatomy in perspective view (560), there is a user's tooth crown point (562) that grips the oral insert keying feature (564) in part to keep the oral insert in place during cleaning. For a treatment plan with oral anatomy shift in perspective view (570), there is a shift in the position of the user's teeth moving the first tooth crown point (562) to a second tooth crown point (572). For the treatment plan keying feature (574) to cover both the first tooth crown point (562) and second tooth crown point (572), a wider keying feature (574) is used to accommodate the tooth crown point in either position (562), (572). As referred to herein, this converts a keying feature point (564) into a keying feature tray (574).

Thus, by being less precise with a tray (574), more flexibility with oral anatomy shift is able to be tolerated and/or the oral insert is more forgiving. In one embodiment, with a treatment plan oral insert, the tray (574) is partially filled with a compliant and/or biocompatible keying material, for example, a soft resin, foam, and/or rubber, that allows the user to better grip with teeth at either tooth crown point location (562), (572) to compensate for the less precise keying feature (574). Not shown in FIG. 5C, if as part of a treatment plan a tooth is removed and/or a tooth crown point is removed, the keying feature itself is removed, referred to herein as a keying absence.

In one embodiment, not shown in FIG. 5C, based on a treatment plan, keying features are only designated for teeth that are not expected to be shifting. For example, last molars may not move in a given treatment plan where premolars are moving and/or rotating. In this scenario, keying features are designed for last molars but not premolars.

In one embodiment, when a treatment plan associated with a user indicates a corresponding amount of movement associated with relative alignment between an upper arch of the user and a lower arch of the user, the oral insert uses virtual articulation, a virtual open bite position scan, and/or the treatment plan arch movement to adjust the relationship between the upper arch and lower arch for the oral insert, as disclosed in U.S. Provisional Patent Application No. 63/591,397 entitled VIRTUAL ARTICULATION OF THE ORAL ANATOMY ASSOCIATED WITH A USER filed Oct. 18, 2023 which is incorporated herein by reference for all purposes, and U.S. patent application Ser. No. 18/908,556 entitled OPEN BITE POSITION SCANNING filed Oct. 7, 2024 which is incorporated herein by reference for all purposes.

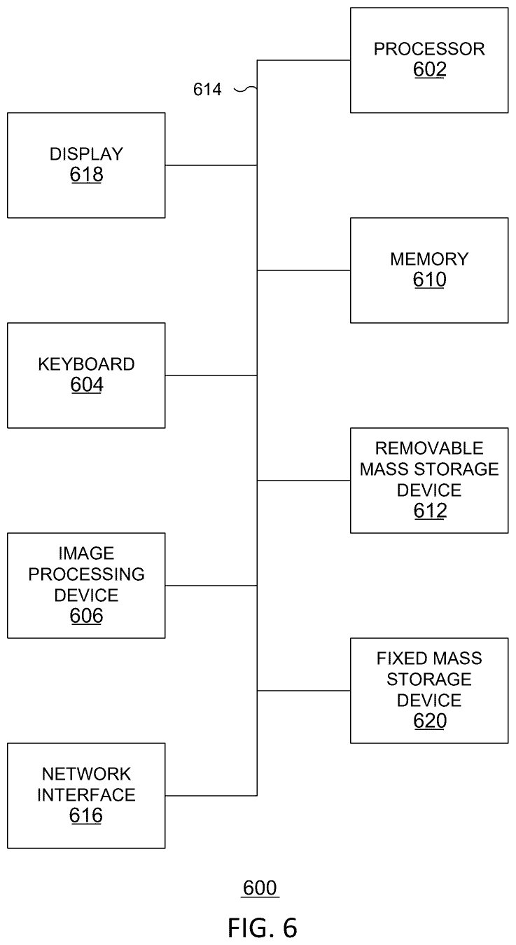

FIG. 6 is a functional diagram illustrating a server for improving the fit and efficacy of an oral health device for a user in which the user's oral anatomy shifts over a period of time in accordance with an embodiment. As will be apparent, other server architectures and configurations can be used to perform the described product generation technique. Server (600), which includes various subsystems as described below, includes at least one microprocessor subsystem (also referred to as a processor or a central processing unit (CPU) (602)). For example, processor (602) can be implemented by a single-chip processor or by multiple processors. In some embodiments, processor (602) is a general purpose digital processor that controls the operation of the server (600). In some embodiments, processor (602) also includes one or more coprocessors or special purpose processors (e.g., a graphics processor, a network processor, etc.). Using instructions retrieved from memory (610), processor (602) controls the reception and manipulation of input data received on an input device (e.g., image processing device (606), I/O device interface (604)), and the output and display of data on output devices (e.g., display (618)).

Processor (602) is coupled bi-directionally with memory (610), which can include, for example, one or more random access memories (RAM) and/or one or more read-only memories (ROM). As is well known in the art, memory (610) can be used as a general storage area, a temporary (e.g., scratch pad) memory, and/or a cache memory. Memory (610) can also be used to store input data and processed data, as well as to store programming instructions and data, in the form of data objects and text objects, in addition to other data and instructions for processes operating on processor (602). Also as is well known in the art, memory (610) typically includes basic operating instructions, program code, data, and objects used by the processor (602) to perform its functions (e.g., programmed instructions). For example, memory (610) can include any suitable computer readable storage media described below, depending on whether, for example, data access needs to be bi-directional or uni-directional. For example, processor (602) can also directly and very rapidly retrieve and store frequently needed data in a cache memory included in memory (610).

A removable mass storage device (612) provides additional data storage capacity for the server (600), and is optionally coupled either bi-directionally (read/write) or uni-directionally (read only) to processor (602). A fixed mass storage (620) can also, for example, provide additional data storage capacity. For example, storage devices (612) and/or (620) can include computer readable media such as magnetic tape, flash memory, PC-CARDS, portable mass storage devices such as hard drives (e.g., magnetic, optical, or solid state drives), holographic storage devices, and other storage devices. Mass storages (612) and/or (620) generally store additional programming instructions, data, and the like that typically are not in active use by the processor (602). It will be appreciated that the information retained within mass storages (612) and (620) can be incorporated, if needed, in standard fashion as part of memory (610) (e.g., RAM) as virtual memory.

In addition to providing processor (602) access to storage subsystems, bus (614) can be used to provide access to other subsystems and devices as well. As shown, these can include a display (618), a network interface (616), an input/output (I/O) device interface (604), an image processing device (606), as well as other subsystems and devices. For example, image processing device (606) can include a camera, a scanner, etc.; I/O device interface (604) can include a device interface for interacting with a touchscreen (e.g., a capacitive touch sensitive screen that supports gesture interpretation), a microphone, a sound card, a speaker, a keyboard, a pointing device (e.g., a mouse, a stylus, a human finger), a Global Positioning System (GPS) receiver, an accelerometer, and/or any other appropriate device interface for interacting with system (600). Multiple I/O device interfaces can be used in conjunction with server (600). The I/O device interface can include general and customized interfaces that allow the processor (602) to send and, more typically, receive data from other devices such as keyboards, pointing devices, microphones, touchscreens, transducer card readers, tape readers, voice or handwriting recognizers, biometrics readers, cameras, portable mass storage devices, and other computers.

The network interface (616) allows processor (602) to be coupled to a 3D printer, another computer, one or more robotic systems, a computer network, a network of storage bins, or a telecommunications network using a network connection as shown. For example, through the network interface (616), the processor (602) can receive information (e.g., data objects or program instructions) from another network, or output information to another network in the course of performing method/process steps. Information, often represented as a sequence of instructions to be executed on a processor, can be received from and outputted to another network. An interface card or similar device and appropriate software implemented by (e.g., executed/performed on) processor (602) can be used to connect the server (600) to an external network and transfer data according to standard protocols. For example, various process embodiments disclosed herein can be executed on processor (602), or can be performed across a network such as the Internet, intranet networks, or local area networks, in conjunction with a remote processor that shares a portion of the processing. Additional mass storage devices (not shown) can also be connected to processor (602) through network interface (616).

In addition, various embodiments disclosed herein further relate to computer storage products with a computer readable medium that includes program code for performing various computer-implemented operations. The computer readable medium includes any data storage device that can store data which can thereafter be read by a server. Examples of computer readable media include, but are not limited to: magnetic media such as disks and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code as produced, for example, by a compiler, or files containing higher level code (e.g., script) that can be executed using an interpreter.

The server shown in FIG. 6 is but an example of a server suitable for use with the various embodiments disclosed herein. Other servers suitable for such use can include additional or fewer subsystems. In some servers, subsystems can share components (e.g., for touchscreen-based devices such as smart phones, tablets, etc., I/O device interface (604) and display (618) share the touch sensitive screen component, which both detects user inputs and displays outputs to the user). In addition, bus (614) is illustrative of any interconnection scheme serving to link the subsystems. Other computer architectures having different configurations of subsystems can also be utilized.

Although the foregoing embodiments have been described in some detail for purposes of clarity of understanding, the invention is not limited to the details provided. There are many alternative ways of implementing the invention. The disclosed embodiments are illustrative and not restrictive.

1. A method, comprising:
receiving a treatment plan associated with a user;
designing a plurality of oral inserts based on the treatment plan associated with the user, wherein the plurality of oral inserts include a corresponding plurality of fluid nozzles, wherein a corresponding location of one, some, or all of a first plurality of fluid nozzles associated with a first oral insert of the plurality of oral inserts is different than a corresponding location of one, some, or all of a second plurality of fluid nozzles associated with a second oral insert of the plurality of oral inserts; and
generating the plurality of oral inserts.

2. The method of claim 1, wherein the treatment plan associated with the user indicates at least one of the following: a series of steps for one, some, or all of the user's teeth; and a corresponding amount of movement associated with one, some, or all of the user's teeth.

3. The method of claim 2, wherein the corresponding amount of movement associated with the one, some, or all of the user's teeth includes linear movement, angular movement, rotational movement, a removal of teeth, and/or a change in number of teeth.

4. The method of claim 1, wherein the first oral insert of the plurality of oral inserts is designed for a subset of a plurality of steps of the treatment plan associated with the user.

5. The method of claim 4, wherein the subset of the plurality of steps of the treatment plan associated with the user comprises a set of movement steps associated with one, some, or all of the user's teeth, and wherein the first oral insert of the plurality of oral inserts is designed for a flexible keying feature.

6. The method of claim 5, wherein the flexible keying feature comprises at least one of the following: a keying tray; a compliant keying material; and a keying absence.

7. The method of claim 4, wherein the subset of the plurality of steps of the treatment plan associated with the user comprises a set of movement steps associated with one, some, or all of the user's teeth, and wherein the first oral insert of the plurality of oral inserts is designed for a flexible fluid nozzle.

8. The method of claim 7, wherein the flexible fluid nozzle comprises at least one of the following: a less precise nozzle shape; a more broad nozzle spray; a nozzle absence in an event that the subset of the plurality of steps of the treatment plan associated with the user comprises a tooth removal; and a nozzle addition in an event that the subset of the plurality of steps of the treatment plan associated with the user comprises a tooth emergence.

9. The method of claim 1, wherein the treatment plan associated with the user indicates a corresponding amount of movement associated with relative alignment between an upper arch of the user and a lower arch of the user.

10. The method of claim 1, wherein the treatment plan associated with the user indicates a rinse adjustment comprising at least one of the following: a treatment rinse solution, an after treatment rinse solution, a rinse duration setting, a rinse intensity setting, and a rinse pulsation setting.

11. The method of claim 1, wherein the first oral insert of the plurality of oral inserts is designed for a sub-step of a plurality of steps of the treatment plan associated with the user.

12. The method of claim 1, wherein at least one of the corresponding plurality of fluid nozzles is at least one of the following: directed downwards towards a gingival margin, an interproximal space between the user's teeth, an incisal edge, or contours of occlusal, facial, lingual, mesial, and distal surfaces of the user's teeth; directed upwards towards a gingival margin, an interproximal space between the user's teeth, an incisal edge, or contours of occlusal, facial, lingual, mesial, and distal surfaces of the user's teeth; directed towards a gingival margin, an interproximal space between the user's teeth, an incisal edge, or contours of occlusal, facial, lingual, mesial, and distal surfaces of the user's teeth at a polar angle between 0° and 180°; and directed towards a gingival margin, an interproximal space between the user's teeth, an incisal edge, or contours of occlusal, facial, lingual, mesial, and distal surfaces of the user's teeth at an azimuthal angle between 0° and 180°.

13. The method of claim 1, wherein at least one of the corresponding plurality of fluid nozzles is at least one of the following: directed downwards towards a bracket, wire, or attachment associated with a tooth of the user; directed upwards towards a bracket, wire, or attachment associated with a tooth of the user; directed towards a bracket, wire, or attachment associated with the user at a polar angle between 0° and 180°; and directed towards a bracket, wire, or attachment associated with the user at an azimuthal polar angle between 0° and 180°.

14. The method of claim 1, wherein the first oral insert of the plurality of oral inserts includes a first manifold having a first number of fluid nozzles and the second oral insert of the plurality of oral inserts includes the first manifold having a second number of fluid nozzles, wherein the first number of fluid nozzles is different than the second number of fluid nozzles.

15. The method of claim 13, wherein the first manifold of the second oral insert includes one or more flow balancing ports.

16. The method of claim 1, wherein a shape associated with the corresponding plurality of fluid nozzles is the same.

17. The method of claim 1, wherein a shape associated with at least one of the corresponding plurality of fluid nozzles is different than other fluid nozzles of the corresponding plurality of fluid nozzles.

18. The method of claim 1, wherein a location of a first set of one or more keying features associated with the first oral insert is different than at least one of one or more other oral inserts of the plurality of oral inserts.

19. A system, comprising:
a processor configured to:
receive a treatment plan associated with a user;
design a plurality of oral inserts based on the treatment plan associated with the user, wherein the plurality of oral inserts include a corresponding plurality of fluid nozzles, wherein a corresponding location of one, some, or all of a first plurality of fluid nozzles associated with a first oral insert of the plurality of oral inserts is different than a corresponding location of one, some, or all of a second plurality of fluid nozzles associated with a second oral insert of the plurality of oral inserts; and
generate the plurality of oral inserts; and
a memory coupled to the processor and configured to provide the processor with instructions.

20. A computer program product embodied in a non-transitory computer readable medium and comprising computer instructions for:
receiving a treatment plan associated with a user;
designing a plurality of oral inserts based on the treatment plan associated with the user, wherein the plurality of oral inserts include a corresponding plurality of fluid nozzles, wherein a corresponding location of one, some, or all of a first plurality of fluid nozzles associated with a first oral insert of the plurality of oral inserts is different than a corresponding location of one, some, or all of a second plurality of fluid nozzles associated with a second oral insert of the plurality of oral inserts; and
generating the plurality of oral inserts.
